(19) United States
(12) Patent Application Publication
(10) Pub. No.: US 2006/0084088 A1
(43) Pub. Date: Apr. 20, 2006

(54) Tracking biological samples and their processing history

(76) Inventors: Emily R. Schultz, Bothell, WA (US); Jesse Chan, Bothell, WA (US); Pamela Po-Ming Lui Griffith, Bellevue, WA (US); Aaron Stuart Cunningham, Bothell, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/187,163

(22) Filed: Jul. 22, 2005

Related U.S. Application Data
(60) Provisional application No. 60/619,347, filed on Oct. 15, 2004.

(51) Int. Cl. C12Q 1/68; G06F 19/00; C12M 1/34

(52) U.S. Cl. 435/6; 702/20; 435/287.2

(57) ABSTRACT

Biological samples undergo drastic changes during the execution of an experiment to aid in the determination of gene discovery, disease diagnosis, drug discovery, toxicological research, and so on. After a biological sample is processed, pieces of information about the state of a biological sample are generated. Pedigree information is also generated when a biological sample is sub-divided into multiple portions or when multiple biological samples are combined. These pieces of information illuminate the experiment process in a way that can help scientists to better understand the failure or the success of an experiment. Various pieces of information, such as the state and the pedigree of biological samples are tracked, traced, or searched, so as to allow scientists to piece together a picture of greater experimental clarity.

### CROSS-REFERENCE TO A RELATED APPLICATION

This application claims the benefit of U.S. Provisional Application No. 60/619,347, filed Oct. 15, 2004, which is incorporated herein by reference.

### FIELD OF THE INVENTION

The present invention relates generally to biological sample history, and more particularly, to the tracking, tracing, or searching laboratory events that form biological samples, such as genetic materials or protein materials, and their processing history.

### BACKGROUND OF THE INVENTION

Biotechnology increasingly relies upon the preparation and analysis of numerous biological or chemical samples. For example, it may be necessary to screen thousands of candidate drugs for a desired biological activity by analyzing changes in gene expression caused by each of the drug candidates. Various systems, such as a microarray system 100, can be used to aid the experimental manipulation of biological samples. During an experiment, portions of genetic samples 102 can be placed on a container, such as a plate 104, which has multiple wells that hold these portions of genetic samples 102. See FIG. 1. (The plate is visually presented as a rectangular form with a number of circles, each circle representing a well.) An experiment typically has many steps. With each step, portions of the genetic samples 102 undergo drastic processing in order for scientists to discover whether a biological theory is correct or what the results of an application of a new drug would be.

As an example of processing, each amount of the genetic samples 102 can be sub-divided into multiple portions. These multiple portions can be placed in multiple wells in a plate 106 during the next step as specified by the requirements of the experiment. Enzymes, such as any of the numerous complex proteins that are produced by living cells and catalyze specific biochemical reactions at certain temperatures, or a reagent, which is a substance used to cause a particular chemical or biological activity, can be added to one or more wells on the plate 106 that contain portions of the sub-divided amount of a genetic sample from a well on the plate 104. Once an amount of the genetic samples 102 has been sub-divided and changed by the enzymes or reagent, the amount is transformed, and no process can reverse this transformation. Moreover, instead of being sub-divided, portions of different genetic samples may be combined and processed into a new biological sample, which can be stored in a single well on a plate 108 where further enzymes or reagents or even additional biological materials may be added to that well. This combination process, like the sub-divided process above, is irreversible.

To better understand the progression of an experiment, a conventional laboratory information management system 110 is used to control and record data as the experiment is being executed in a laboratory. Biological sample containers, such as plates 104-108, are typically labeled by barcodes, and these barcodes are continuously referenced by the conventional laboratory information management system 110 as biological samples in the plates 104-108 undergo the execution of various steps of an experiment protocol, which may require changes to the biological samples that are placed in one or more wells or plates 104-108. Conventional laboratory information management system 110 can track the biological sample containers via identifiers, such as barcodes, but it completely lacks an ability to track the history and pedigree of each biological sample in an experiment in the biological sample containers for an experiment.

As long as a biological sample has not changed or been removed from the container being tracked, conventional laboratory information management system 110 can provide much information about that particular sample. As each step in an experiment protocol is executed, conventional laboratory information management system 110 can track the well and therefore the sample because there is a rigid one-to-one correspondence between the biological sample and the well containing the biological sample. But in practice, biological samples are taken from one container, such as a well, and placed into another well. But every time the biological sample is processed, it is no longer the original biological sample due to the addition of enzymes, reagents, or other biological materials. This is particularly important to note when an experiment has failed or had a questionable result.

There are many reasons why an experiment result may fail or otherwise give rise to a questionable result, such as a malfunction of laboratory machinery; mislabeling of samples; overamplification of particular reagents or enzymes; and so on. Without knowing the history of biological samples, it may be impossible for scientists to decompose the experiment and find out what has gone right and the point at which the experiment has gone wrong, and may eventually cause scientists to no longer trust the system 100 to provide a desired experiment experience that can produce information for biological analysis. As a result, usage of the system 100 will diminish in the marketplace. Thus, there is a need for a system, method, and computer-readable medium for tracking the history of biological samples while avoiding or reducing the foregoing and other problems associated with existing systems.

### SUMMARY OF THE INVENTION

In accordance with this invention, a system, method, and computer-readable medium for tracking, tracing, or searching representations of biological samples and their processing history is provided. The system form of the invention includes a system for executing biological experiments. The system comprises a container for containing a biological sample. The container includes an identifier that is capable of being scanned and computer-readable to identify the container. The system also comprises a sample tracking facility that tracks the biological sample and the container such that various pieces of information in connection with the biological sample are trackable independent of the container that contains the biological sample.

In accordance with further aspects of this invention, the system form of the invention includes a biological sample tracking system. The system comprises a user interface for entering, modifying, or querying the pedigree of a biological sample. The system further comprises a database for storing the history of the transformation of the biological sample. The database also includes a first table set for storing information that pertains to the splitting of the biological sample into multiple portions. The database further includes a second table set for storing information that pertains to a pool of the biological sample with another biological sample.

In accordance with further aspects of this invention, the system form of the invention includes a system for tracking biological samples. The system comprises a number of tracking entities for representing the types of container for containing biological samples and the types of processing of biological samples. The system further comprises a number of tables in a database for storing the history of the processing of biological samples. The tables includes a first table set for storing identifying information about a biological sample and identifying information about the parent of the biological sample.

In accordance with further aspects of this invention, the computer-readable medium form of the invention includes a computer-readable medium having one or more data structures stored thereon for use by a computing system to track the history of biological samples. The data structures comprise a sample unit class for defining attributes and services connected with a discrete amount of biological sample for experimentation under controlled conditions. The data structures further comprise a container class for defining attributes and services connected with a receptacle for holding biological fragments defined by the sample unit class.

In accordance with further aspects of this invention, the computer-readable medium form of the invention includes a computer-readable medium having one or more database tables stored thereon for use by a computing system to track, trace, or search pieces of information connected with biological samples. The database tables comprise a sample database table that includes a sample identifier field that is indicative of a unique identifier of a biological sample that has been instantiated as a record of information in the sample database table. The sample database table further includes a parent table name field that is indicative of a name of a database table from which a parent of the biological sample is instantiated as a record of information. The sample database table also includes a parent key identifier field that is indicative of a unique identifier for identifying the parent of the biological sample, which is an instantiation as a record of information.

In accordance with further aspects of this invention, the method form of the invention includes a method for tracking biological samples. The method comprises preparing source sample for execution of a protocol. The method also comprises recording the history of a sample as a step in the protocol is executed. The method additionally comprises reporting the history of the sample to a user upon request by the user.

In accordance with further aspects of this invention, the computer-readable medium form of the invention includes computer-executable instructions stored thereon that implements a method for tracking biological samples. The method comprises preparing source sample for execution of a protocol. The method also comprises recording the history of a sample as a step in the protocol is executed. The method further comprises reporting the history of the sample to a user upon request by the user.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram illustrating a conventional laboratory information management system for controlling and recording experiments involving biological samples;

FIG. 2A is a block diagram illustrating an exemplary sample tracking facility that tracks not only containers but also biological samples and their processing history;

FIG. 2B is a block diagram illustrating changes that may occur to a biological sample during processing in accordance with one embodiment of the present invention;

FIG. 3A is a block diagram illustrating an exemplary system for tracking the biological samples and their processing history;

FIG. 3B is a class diagram illustrating classes or user-defined types for implementing a tracking facility in accordance with one embodiment of the present invention;

FIG. 3C is a data structure diagram illustrating tables implementable on a database forming a tracking facility in accordance with one embodiment of the present invention; and

FIGS. 4A-4K are process diagrams illustrating a method for tracking biological samples and their processing history.

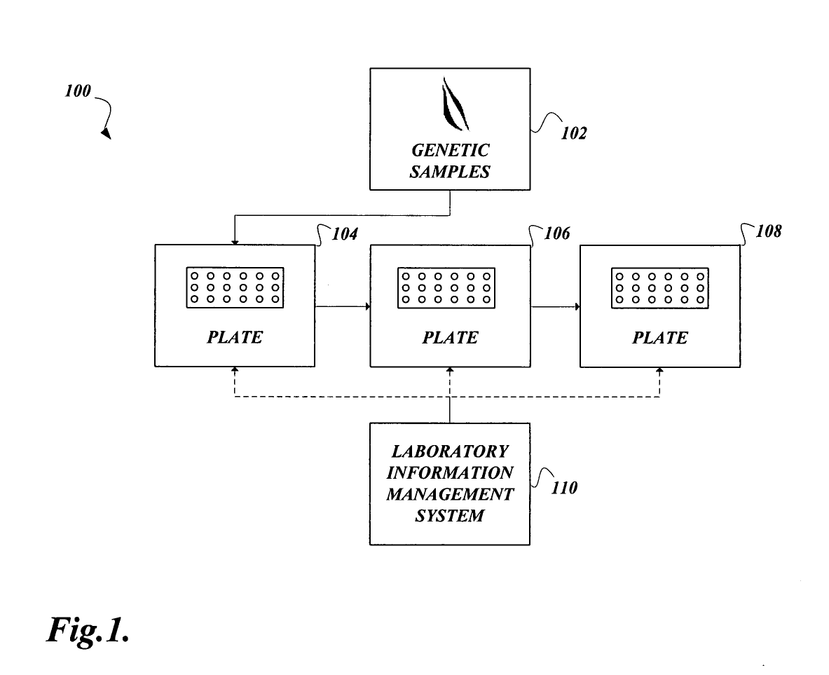

### DETAILED DESCRIPTION OF THE PREFERRED EMBODIMENT

Biological samples undergo changes during the execution of an experiment (e.g. microarray experiments) to aid, for example, in the determination of gene discovery, disease diagnosis, drug discovery, toxicological research, and so on. After a process step, pieces of information about the biological sample and its processing history are generated. For example, pedigree information is generated when a biological sample is sub-divided into multiple portions or when multiple biological samples are combined. These pieces of information illuminate the experiment process in a way that can help scientists to better understand the failure or the success of an experiment. Yet, conventional systems lack the facility to track such pieces of information. Various embodiments of the present invention allow biological samples and their processing history to be tracked, traced, or searched, so as to allow scientists to piece together a picture of greater experimental clarity.

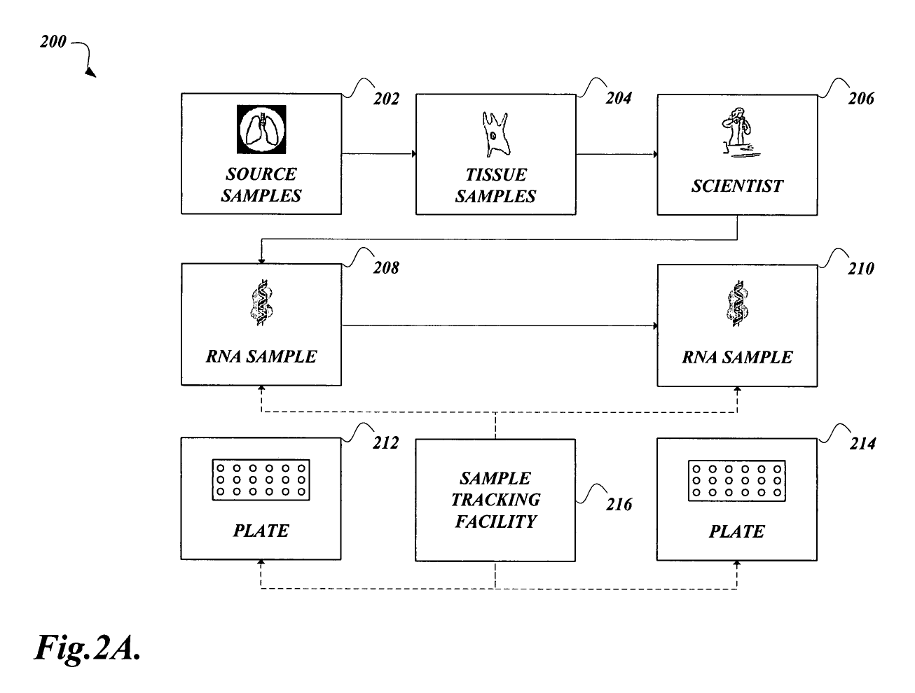

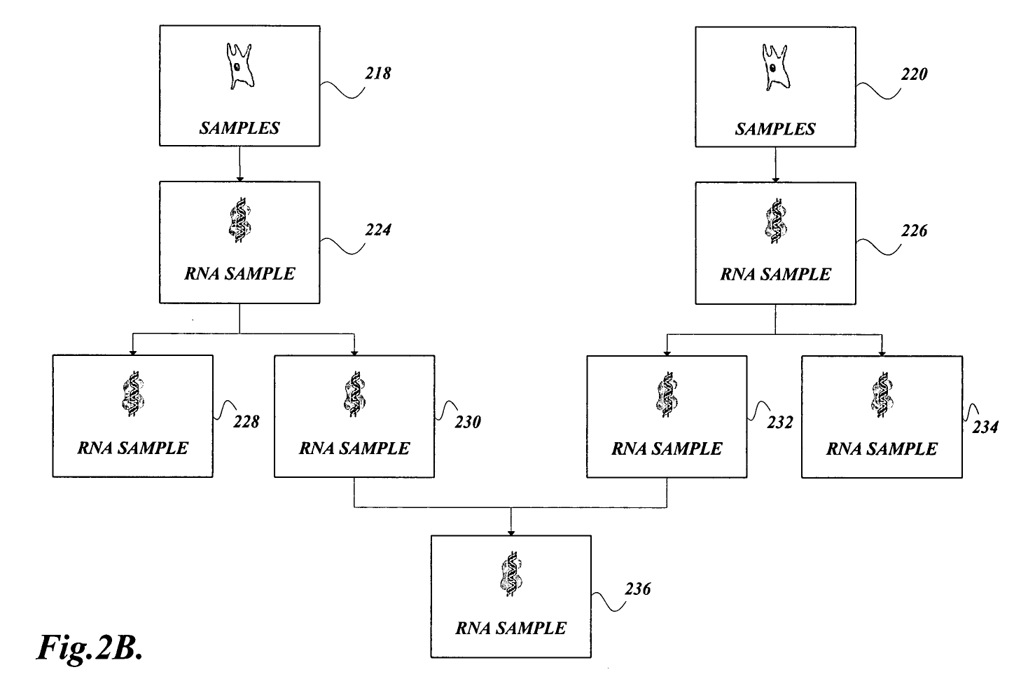

FIG. 2A illustrates a system 200 in which source samples 202, such as lung organs that have been exposed to a new drug of interest, are processed to produce tissue samples 204 by a scientist 206. Genetic materials, such as deoxyribonucleic acid (DNA) or ribonucleic acid (RNA) can be taken from tissue samples 204 by the scientist 206. The term “biological sample” as used hereinabove and hereinbelow means the inclusion of deoxyribonucleic acid (DNA), ribonucleic acid (RNA), tissues, lysates, or protein. These genetic materials, such as RNA samples 208, 210, can be placed on containers, such as plates 212, 214. Various embodiments of the present invention provide a sample tracking facility 216 that tracks not only containers, such as plates 212, 214, but also biological samples, such as RNA samples 208, 210. The sample tracking facility 216 can track the state, condition of RNA samples 208, 210. Additionally, the pedigree, lineage, or the origin and the history of RNA samples 208, 210 can also be tracked by the sample tracking facility 216, hence facilitating better understanding of the recorded sample information during an experiment. Furthermore, other pieces of information in connection with the biological sample (e.g., the RNA samples 208, 210) and its processing history are recorded, such as sample pedigree (e.g., source biological sample or combination of biological samples, and so on); sample transformational state (e.g., rehydrated, purified, and so on); sample processing stage (e.g., in reverse transcription “RT” reaction, hybridizing, and so on); sample quantification data (e.g., concentration, volume, and so on); sample status (e.g., pass/fail based on qualitiative data, redo, and so on); sample location in containers (e.g., a particular well and so on); items applied to the biological sample (e.g., chemical part number, chemical lot, and so on); and equipment applied to the biological sample (e.g., thermocycler, robot, and so on).

FIG. 2B illustrates the changes of biological samples whose changes are better understood by the tracking facility provided by various embodiments of the present invention. RNA samples 224, 226 are portions of genetic materials taken from tissue samples 218, 220. One type of change that can occur to the RNA samples 224, 226 is that RNA samples 224, 226 can be split into multiple portions 228-234. The tracking facility of various embodiments of the present invention provides knowledge that the parentage of RNA samples 228, 230 is RNA sample 224. Similarly, the tracking facility of various embodiments of the present invention can be queried to find out that the parentage of RNA samples 232, 234 is RNA sample 226. The tracking facility can also be queried to understand whether RNA samples 224, 226 have been split into multiple portions and the number of portions thereof. Suppose that the RNA samples 224-236 were used for an experiment A. One or more of the RNA samples 224-236 used in the experiment A can be used for an experiment B and the tracking facility provided by various embodiments of the present invention can track one or more samples participating in multiple unrelated experiments and the progenies of one or more samples.

Another change that can be tracked by the tracking facility of various embodiments of the present invention is when two biological samples, such as RNA samples 230, 232, are combined to form a pool, such as RNA sample 236. The tracking facility can be queried to gain knowledge that RNA sample 230 is one of two parents of RNA sample 236 and RNA sample 232 is one of two parents of the pool forming RNA sample 236. RNA sample 236 could also be queried using the tracking facility of various embodiments of the present invention to understand that it is a pool of two members comprising RNA sample 230 and RNA sample 232. A further change that can be tracked by the tracking facility of various embodiments of the present invention includes a situation where a biological sample is reintroduced into a system for a new, different experiment. The biological sample's participation in the new experiment can be separately recorded so that the system is able to distinguish sample pedigree based on a unique experiment identifier.

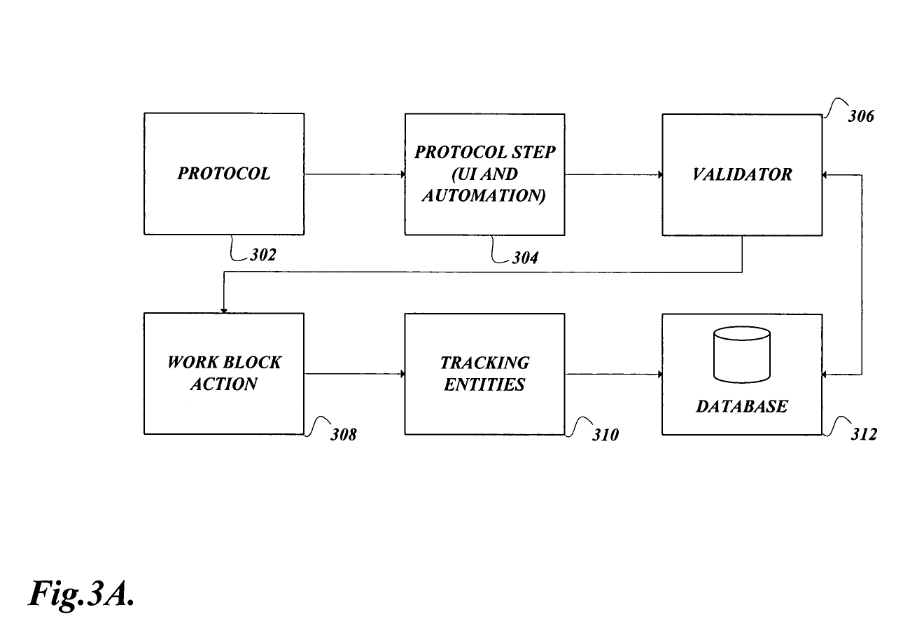

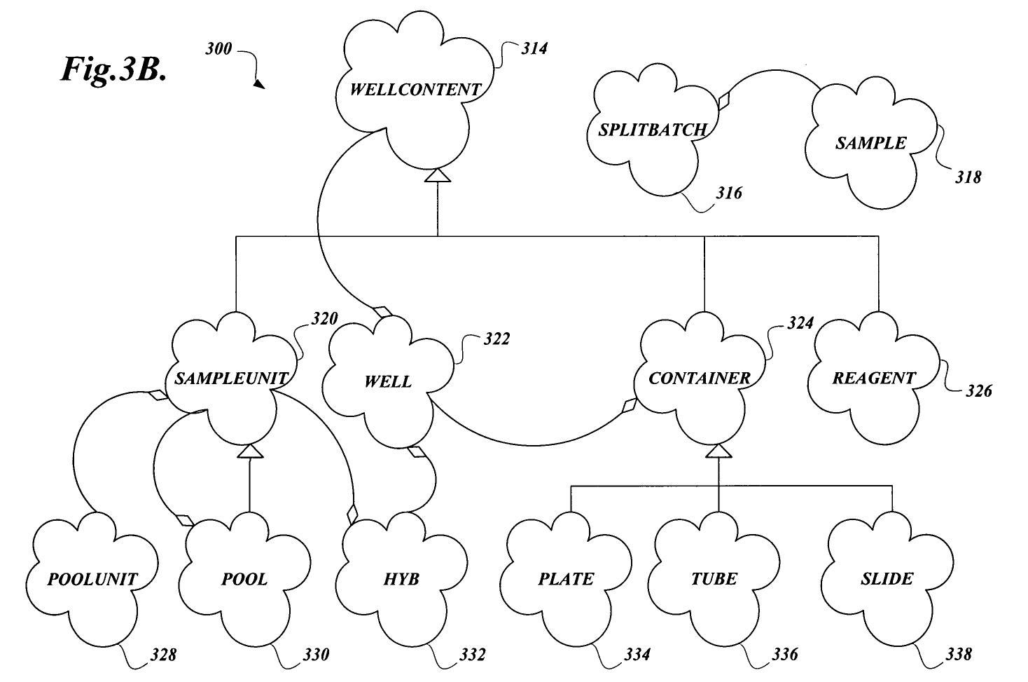

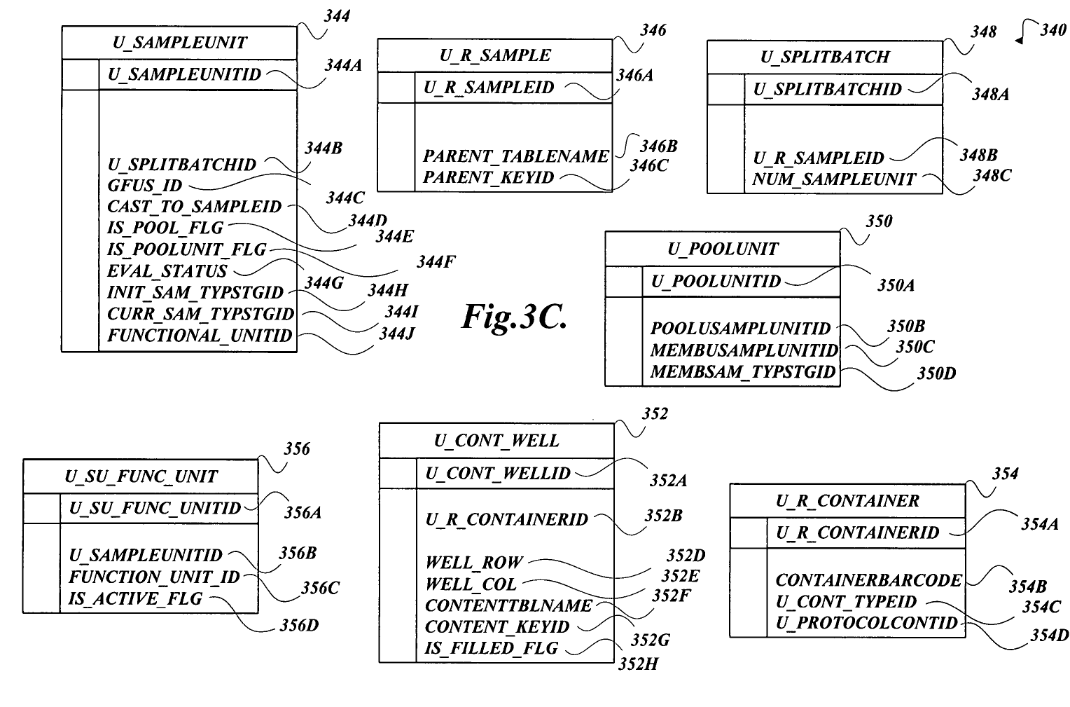

FIG. 3A illustrates a protocol 302, which is a detailed plan of a biological experiment in which the state and pedigree of samples are tracked by a tracking facility in accordance with various embodiments of the present invention. The protocol 302 typically has a number of protocol steps to be executed. A protocol step (which describes both a user interface manually controlled by a user and automation) 304 is a discrete execution unit that executes a particular protocol step in the protocol 302. Prior to the execution of the protocol step 304, information pertaining to a biological sample, such as a bar code identifying a container containing the biological sample, is sent to a validator 306, which can either confirm or deny the validity of such information with a database 312. Any suitable database may be used. One suitable database is a relational database. Another suitable database is an object-oriented database. Various pieces of information include container identities, container types, container processing states, chemical identities, chemical lots, and identities of biological samples. If the database 312 confirms that the information provided by the protocol step 304 is valid, the validator 306 allows the work block action component 308 to be actified. The work block action component 308 executes a protocol step connected with the protocol step 304. In the running of the work block action 308, software entities, such as tracking entities 310, are created to carry out various tasks in the performance of the work block action 308. When the tracking entities 310 need access to data or to record data, the tracking entities 310 communicate with the database 312. Various tracking entities 310 have a corresponding relationship with various database tables stored in the database 312 for accessing and storing data in connection with the performance of the work block action 308.

A system 300 illustrates a class diagram in which each class is a generalized category that describes a group of more specific items, called objects. See FIG. 3B. A class is a descriptive tool used in an object-oriented program to define a set of attributes and/or a set of services (actions available to other parts of the program) that characterize any member (object) of the class. Essentially, each class defines the type of entities it includes and the ways those entities behave.

A sample class 318 defines attributes and services representing a biological sample in the system 300. A splitbatch class 316 defines attributes and services connected with a biological sample that has been divided into multiple portions. In practice, a sample can be used to create many split batches. At the class level, an edge emanating from the sample class 318 and terminating in a rhombus-shaped figure at the splitbatch class 316 indicates that there can be a one-to-one correspondence between an instantiation of a sample class 318 and a splitbatch class 316. A sample can be used to create many splitbatches. A splitbatch instance corresponds to a single sample instance. A wellcontent class 314 defines attributes and services connected with an entity representing content, such as genetic materials, in a well.

A sampleunit class 320 defines attributes and services connected with a discrete amount of a biological sample for experimentation under strictly controlled conditions. Many splitbatches of one or more sampleunits can be created from a single sample. A sample can be a sampleunit that was derived from a parent sample. A splitbatch instantiation is a way to identify all sampleunit instances that were created from a parent sample at the same time. Other sampleunits could exist for the same parent sample, but if they were created at a different time, they would belong to a different splitbatch instance. An edge emanating from the sampleunit class 320 and terminating with a triangular-shaped figure at the wellcontent class 314 indicates that there is an inheritance relationship between the wellcontent class 314 and the sampleunit class 320. In other words, the sampleunit class 320 inherits certain attributes and services from the wellcontent class 314.

A poolunit class 328 defines attributes and services connected with a member of a pool of multiple biological samples. An edge emanating from the poolunit class 328 and terminating with a rhombus-shaped figure at the sampleunit class 320 indicates that for each instantiation of the sampleunit class 320 there can be zero or one instantiation of the poolunit class 328. A pool class 330 defines attributes and services connected with an aggregation of biological samples. An edge emanating from the pool class 330 and terminating with a triangular-shaped figure at the sampleunit class 320 indicates an inheritance relationship between the pool class 330 and the sampleunit class 320 in which the pool class 330 possesses one or more attributes and services of the sampleunit class 320. An edge emanating from the sampleunit class 320 and terminating with a rhombus-shaped figure at the pool class 330 indicates that for each instantiation of the pool class 330 there can be one or more instantiations of the sampleunit class 320.

A well class 322 defines attributes and services connected with an open container for containing biological samples. An edge emanating from the wellcontent class 314 and terminating with a rhombus-shaped figure at the well class 322 indicates that for each instantiation of the well class 322 there can be zero or one instantiation of the wellcontent class 314. A hyb class 332 defines attributes and services connected with a hybridization structure of biological samples. An edge emanating from the hyb class 332 and terminating with a rhombus-shaped figure at the well class 322 indicates that for each instantiation of the well class 322 there can be zero or one instantiation of the hyb class 332. Another edge emanating from the sampleunit class 320 and terminating with a rhombus-shaped figure at the hyb class 332 indicates that for each instantiation of the hyb class 332 there is a single instantiation of the sampleunit class 320. A hyb contains a sample unit but a sample unit may not have an associated hyb.

A container class 324 defines attributes and services connected with a receptacle for holding biological samples. An inheritance relationship between the container class 324 and the wellcontent class 314 is visually established by an edge emanating from the container class 324 and terminating with a triangular-shaped figure at the wellcontent class 314, which represents that the container class 324 possesses one or more attributes and services of the wellcontent class 314. An edge emanating from the well class 322 and ending with a rhombus-shaped figure at the container class 324 indicates that for each instantiation of the container class 324 there can be one or more instantiations of the well class 322. In practice, a container, such as a plate, may have many wells on it, hence the reason for the one to many relationship between the container class 324 and the well class 322.

A plate class 334 defines attributes and services connected with a smooth, flat, thin piece of material on which wells are housed to hold biological samples. An inheritance relationship between the container class 324 and the plate class 334 is established visually by an edge emanating from the plate class 334 and terminating with a triangular-shaped figure at the container class 324, which represents that the plate class 334 possesses one or more attributes and services of the container class 324.

A tube class 336 defines attributes and services connected with a cylindrical structure for holding biological samples. The tube class 336 possesses one or more attributes and/or services of the container class 324, which is visually represented by an edge emanating from the tube class 336 and terminating with a triangular-shaped figure at the container class 324. A slide class 338 defines attributes and services connected with microarrays on which samples are hybridized. The slide class 338 is a subclass of the super-class container class 324 and indicates that the slide class 338 possesses one or more attributes or services of the container class 324. This relationship between the slide class 338 and the container class 324 is represented by an edge emanating from the slide class 338 and terminating with a triangular-shaped figure at the container class 324.

A reagent class 326 defines attributes and services associated with a substance used as an additive to cause chemical or biological activities in a biological sample. An inheritance relationship is established between the reagent class 326 and the wellcontent class 314, which is visually illustrated by an edge emanating from the reagent class 326 and terminating with a triangular-shaped figure at the wellcontent class 314. In other words, the reagent class 326 possesses one or more attributes or services of the wellcontent class 314.

FIG. 3C illustrates a portion of a collection of database tables 344-354, collectively referenced as a schema 340. Each database table 344-354 is basically a file composed of records, each containing fields together with a set of operations for searching, sorting, recombining, and performing other database functions. Each record of database tables 344-354 has a data structure, which is characterized by rows and columns with data occupying or potentially occupying each cell formed by a row-column intersection. Information regarding the biological samples and the history of processing the biological samples therefore has a data structure for describing such information. Many pieces of information are stored by the database besides those mentioned hereinabove, such as sample pedigree, transformational state, processing stage, quantification data, sample status, sample location, chemical items applied to the biological sample, and equipment applied to the biological sample. A portion of such a data structure is illustrated by the schema 340 as shown in FIG. 3C.

The schema 340 includes a U_SAMPLEUNIT table 344; a U_R_SAMPLE table 346; U_SPLITBATCH table 348; a U_POOLUNIT table 350; a U_CONT_WELL table 352; and a U_R_CONTAINER table 354. Each table 344-354 has a number of fields. Information regarding the state and pedigree of samples is stored in these fields which form the columns of a table with information occupying the rows. These tables 344-354 facilitate searches by using data in specified columns in one table to find additional data in another table. Information is matched from a field in one table with information in a corresponding field of another table to produce results for queries that combine requested data from both tables. For example, table 344 contains a U_SPLITBATCHID field 344B and table 348 contains a number of fields including a U_SPLITBATCHID field 348A. The database 312 can match the U_SPLITBATCHID fields 344B, 348A in the two tables 344, 348 to find information (e.g., all biological samples that are children of a particular biological sample). In other words, a database 312 uses matching values in two tables to relate information in one to information in the other.

The U_SAMPLEUNIT table 344 contains a U_SAMPLEUNITID field 344A, which uniquely identifies a sample unit or a discrete amount of biological material; a U_SPLITBATCHID field 344B, which uniquely identifies a split batch or a biological sample that will be or has been divided into multiple portions; a GFUS_ID field 344C, which is a reference to a table that provides a short-cut to find an overall parent identifier for identifying sample units that are essentially from the same biological sample; a CAST_TO_SAMPLEID field 344D, which indicates a child sample instantiated as a row in the table 346 whose parent is a sample unit, and instantiation as a row in the table 344; a IS_POOL_FLG field 344E, which indicates whether a sample unit is a pool of biological samples; a IS_POOLUNIT_FLG field 344F, which indicates whether a sample unit is a member of a pool; an EVAL_STATUS field 344G, which indicates whether the biological sample passes or fails a particular quality control test; a INIT_SAM_TYPSTGID field 344H, which indicates the initial state of a biological sample, such as messenger RNA, ribosomal RNA, transfer RNA, and so on; a CURR_SAM_TYPSTGID field 344I, which indicates the current state of the biological sample, such as messenger RNA, ribosomal RNA, transfer RNA, and so on; and a FUNCTIONAL_UNITID field 344J, which indicates an experiment identifier for identifying a biological sample being used in a particular experiment. Preferably, the initial state includes a sample type of the biological sample (e.g., messenger RNA) and a stage at which the biological sample is at in the processing, among others.

The U_R_SAMPLE table 346 represents a biological sample and contains a U_R_SAMPLEID field 346A, which uniquely identifies a biological sample; a PARENT_TABLENAME field 346B, which indicates the name of the table of the parent of the sample represented by an instantiation or a row in the table 346; and a PARENT_KEYID field 346C, which uniquely identifies a parent of the sample represented by an instantiation or a row of another table.

The U_SPLITBATCH table 348 represents a split batch or a portion of a biological sample and contains a U_SPLITBATCHID field 348A, which uniquely identifies a particular split batch as a row or an instantiation; a U_R_SAMPLEID field 348B, which uniquely identifies a sample represented by an instantiation or row at the table 346; and a NUM_SAMPLEUNIT field 348C, which provides the number of portions divided from a particular sample (which is represented by an instantiation or a row at the table 346).

A U_POOLUNIT table 350 contains a U_POOLUNITID field 350A, which uniquely identifies a pool unit or a member of a pool of biological samples; a POOLUSAMPLUNITID field 350B, which identifies a pool to which the member belongs; a MEMBUSAMPLUNITID field 350C, which identifies the members of a pool of biological samples; and a MEMBSAM_TYPSTGID field 350D, which identifies a sample type for each member of a pool.

A U_CONT_WELL table 352 expresses a well belonging to a container and contains a U_CONT_WELLID field 352A, which uniquely identifies a well; a U_R_CONTAINERID field 352B, which uniquely specifies an instantiation or a row of the table 354 indicating a particular container that contains the well; a WELL_ROW field 352D, which indicates the row of wells at which the well resides in a container; a WELL_COL field 352E, which indicates the column of wells at which the well resides in a container; a CONTTBLNAME field 352F, which indicates the name of a database table containing the instantiation of a sample stored by a well; a CONTENT_KEYID field 352G, which indicates the unique identifier of the content, such as the information stored in the U_SAMPLEUNITID field 344A; and a IS_FILLED_FLG field 352H, which indicates whether there is a biological sample in the well.

A U_R_CONTAINER table 354 expresses a container used in a biological experiment and contains a U_R_CONTAINERID field 354A, which uniquely identifies a container as an instantiation or row in the table 354; a CONTAINERBARCODE field 354B, which stores a container identifier, such as a barcode that is designed to be scanned and read into computer memory for identifying the container; a U_CONT_TYPEID field 354C, which identifies the type of container, such as a plate, a test tube, or a slide, and so on; and a U_PROTOCOLCONTID field 354D, which indicates a particular container used within in a protocol for a specific container.

A U_SU_FUNC_UNIT table 356 contains a U_SU_FUNC_UNITID field 344A, which uniquely identifies a sample unit (or a discrete amount of biological) within a particular experiment; a U_SAMPLEUNITID field 356B, which stores a uniquely identifier for the sample unit or a discrete amount of biological material under the particular experiment; a FUNCTION_UNIT_ID field 356C, which identifies uniquely the particular experiment; and a IS_ACTIVE_FLG field 356D, which indicates various flags associated with the particular experiment.

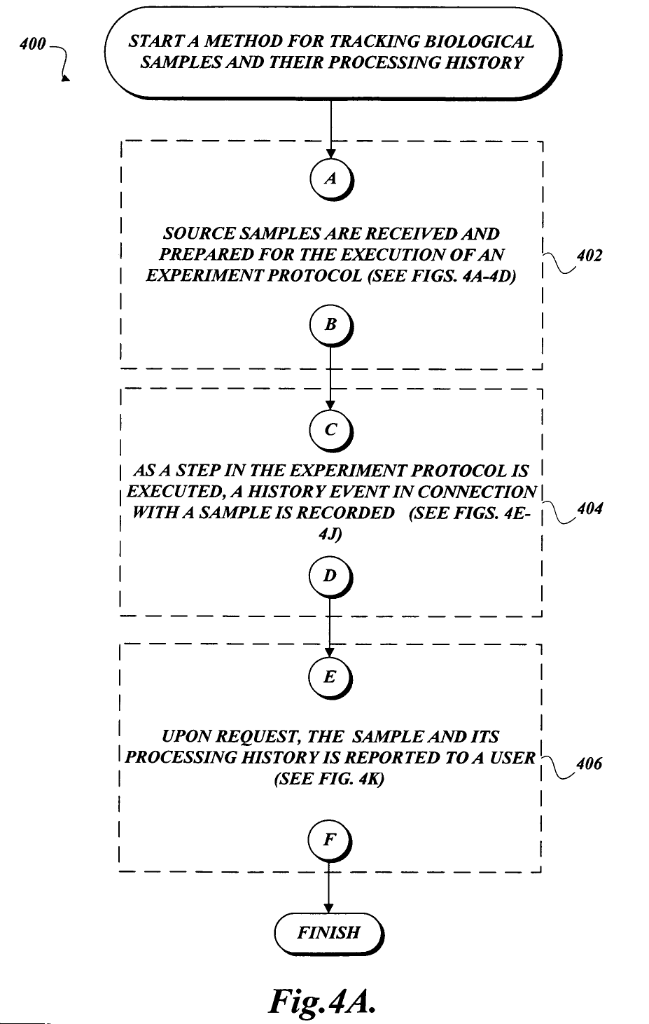

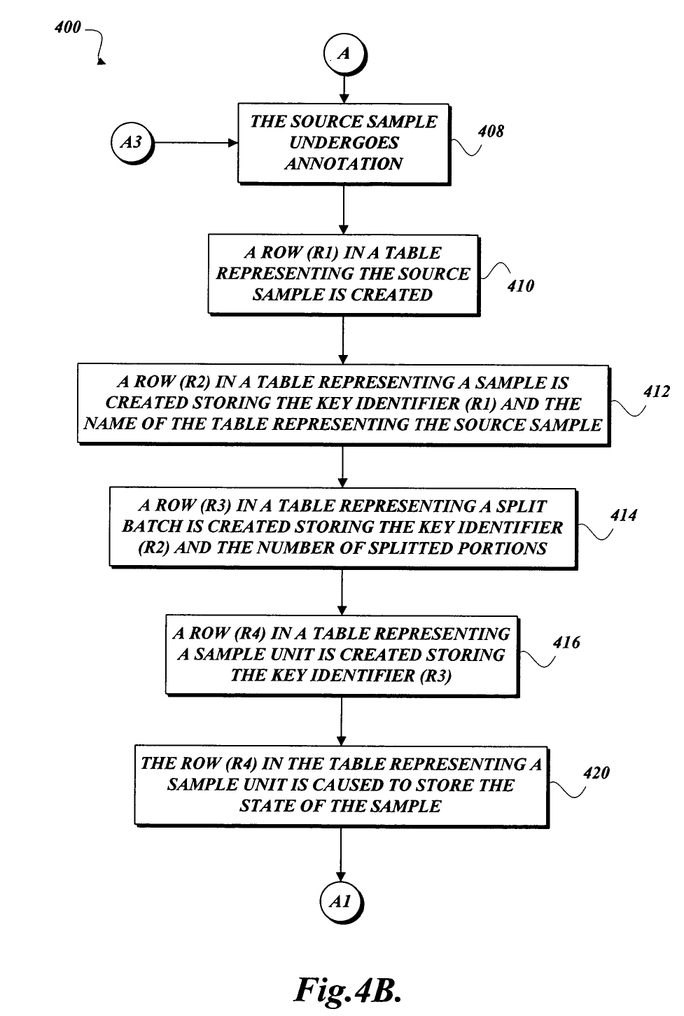

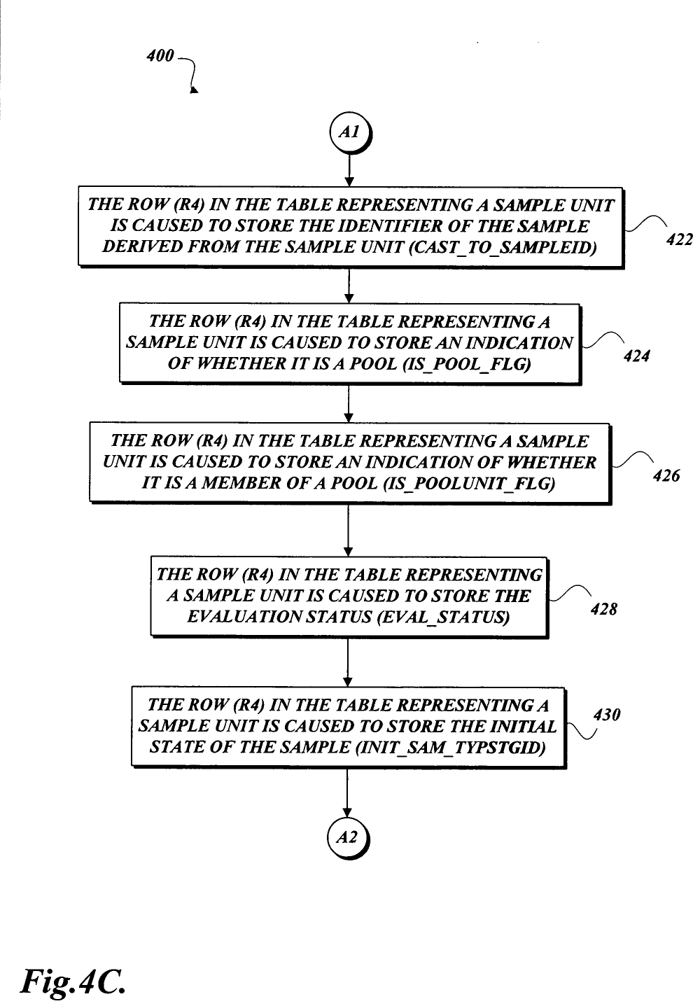

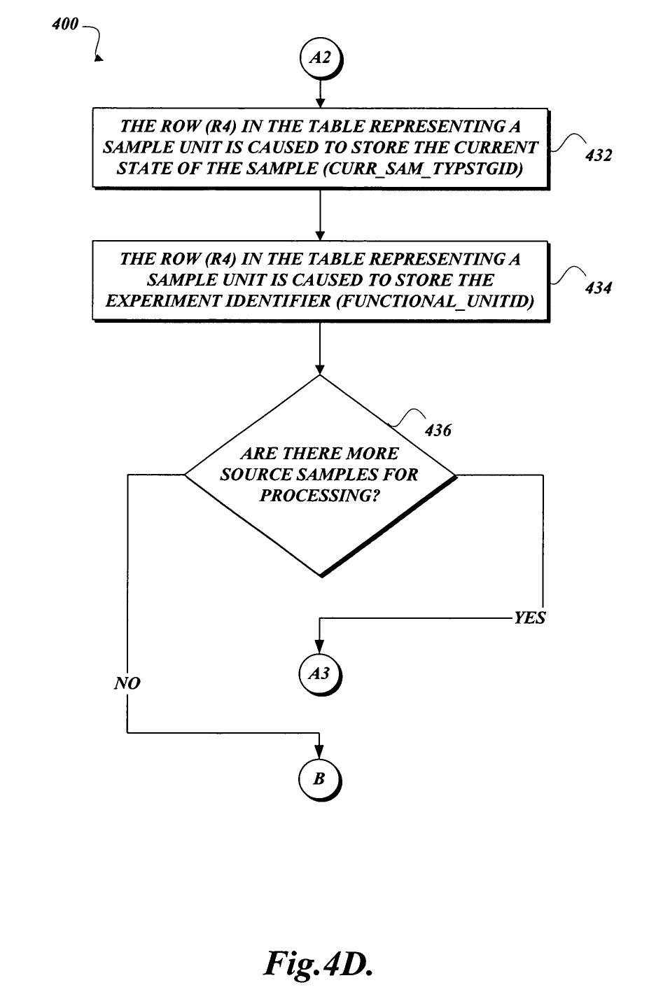

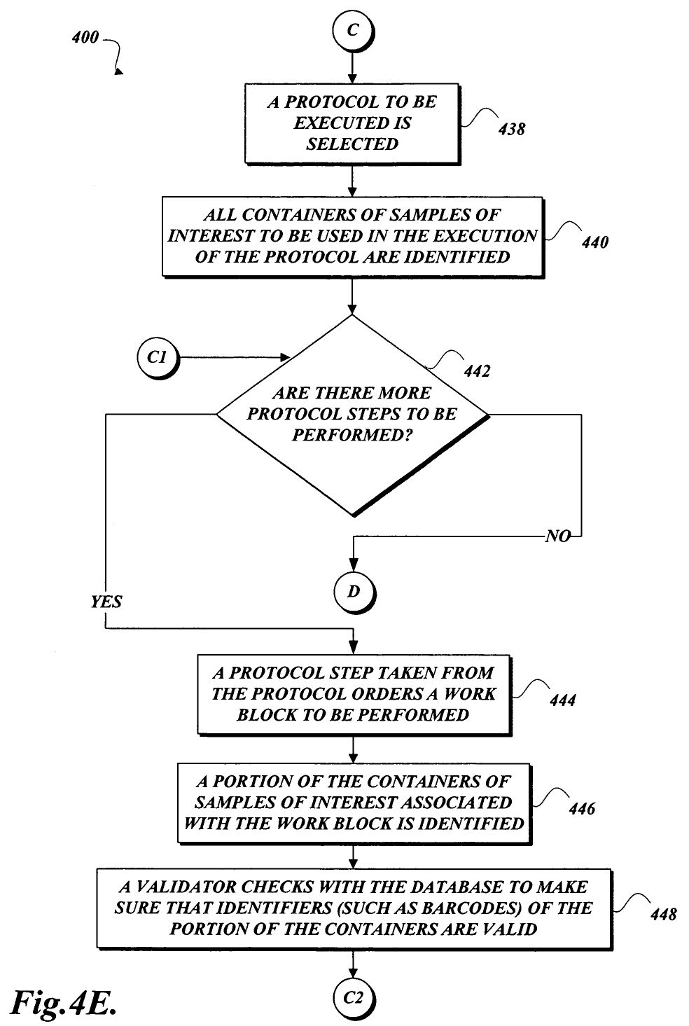

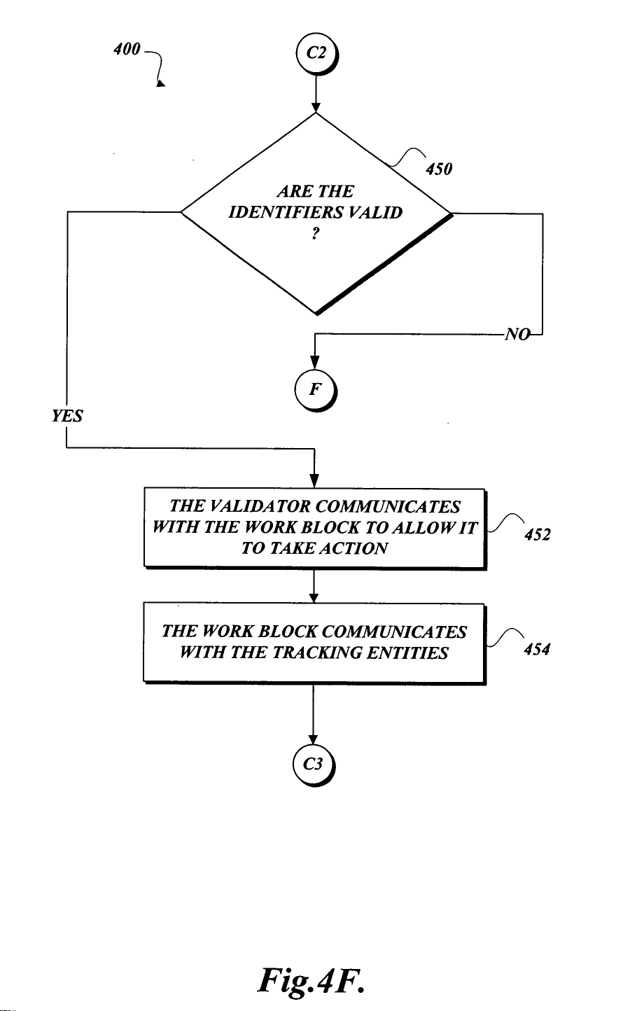

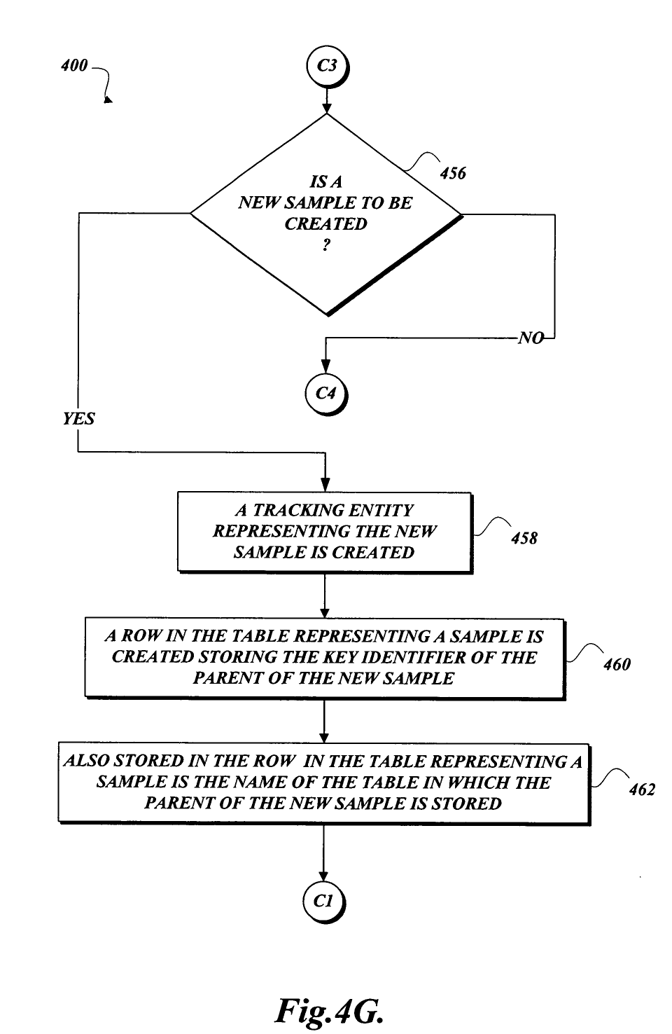

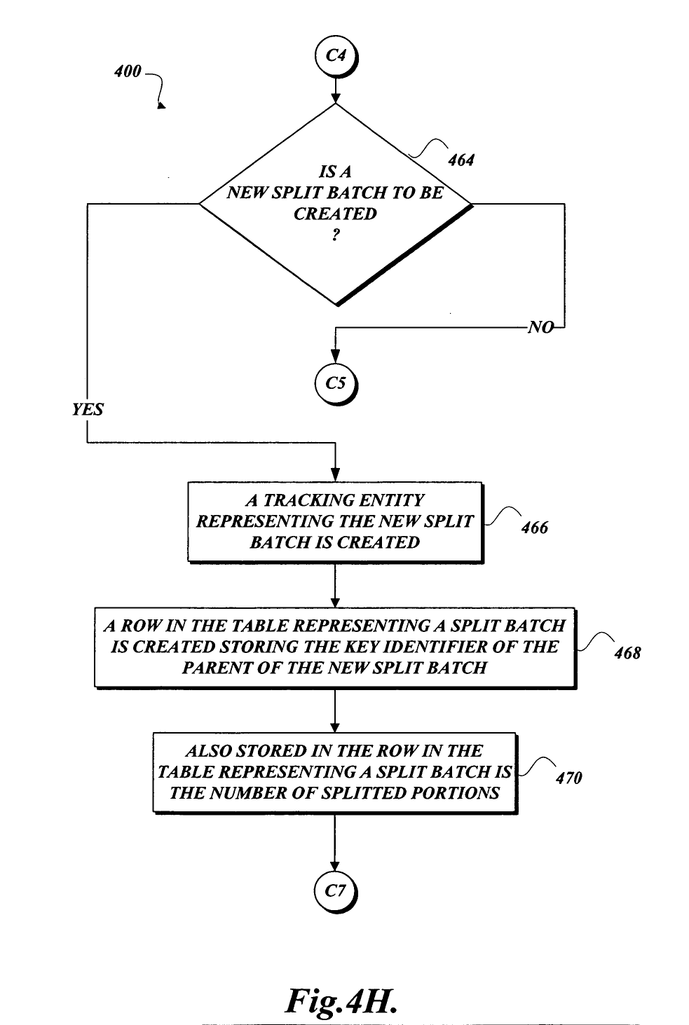

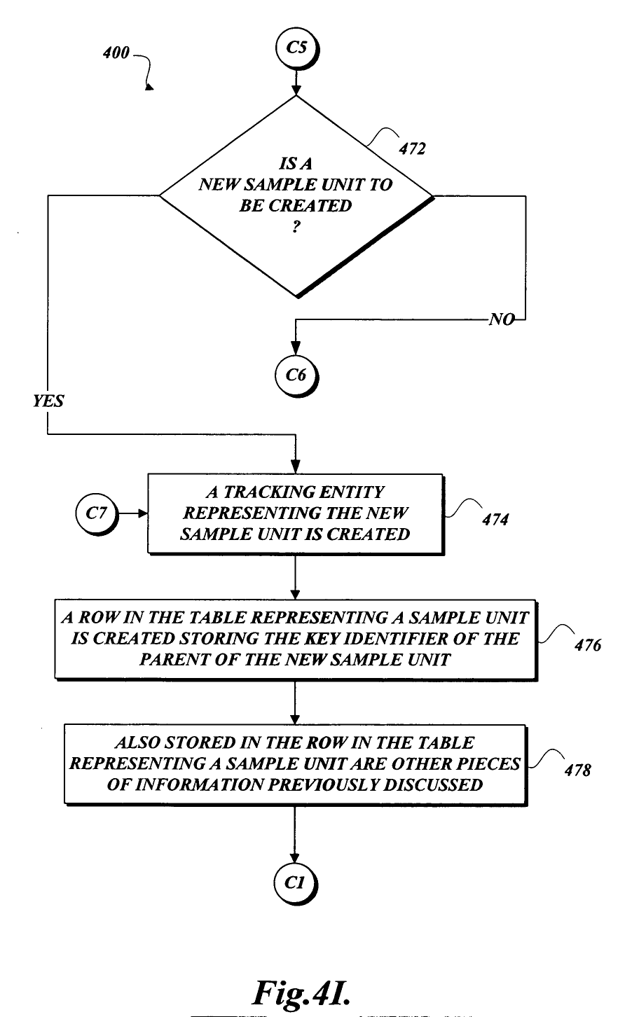

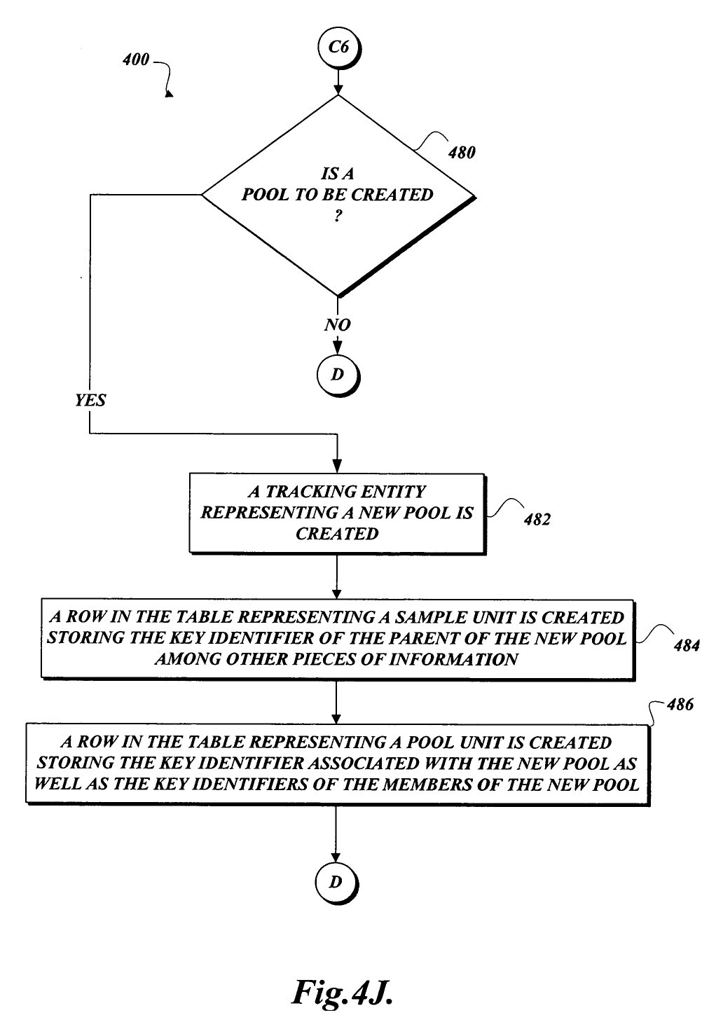

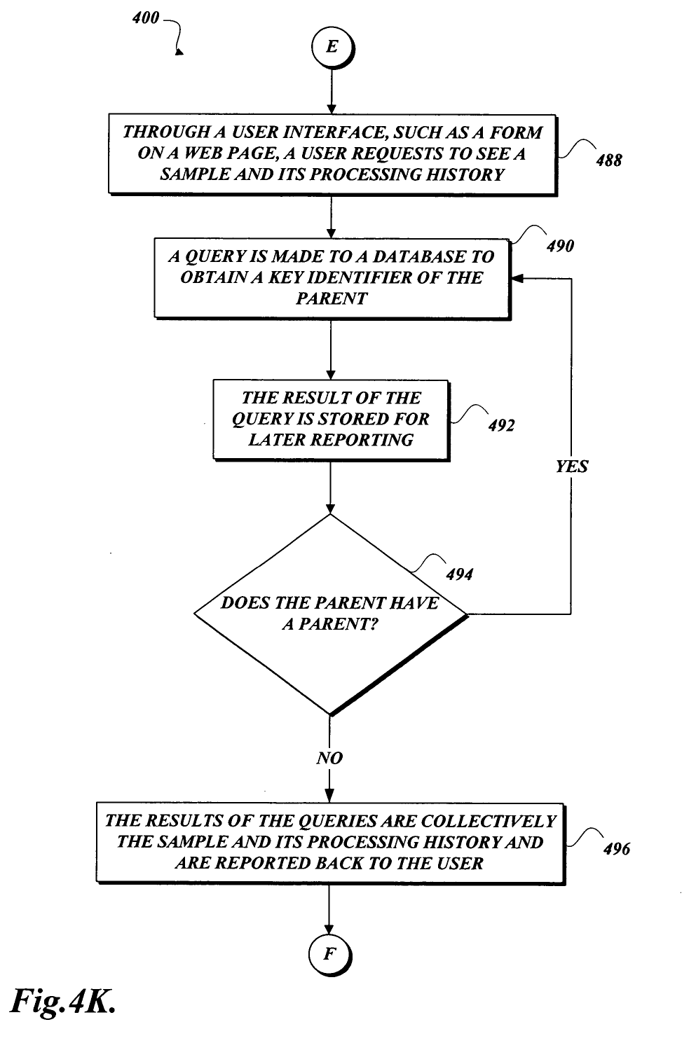

FIGS. 4A-4K illustrate a method 400 for tracking biological samples and their processing history. For clarity purposes, the following description of the method 400 makes references to various elements illustrated in connection with the protocol 302, protocol step 304, validator 306, work block action 308, tracking entities 310, and a database 312 (all of FIG. 3A); class diagram 300 (FIG. 3B); and tables 344-354 illustrated in connection with the schema 340 (FIG. 3C). From a start block, the method 400 proceeds to a set of method steps 402, defined between a continuation terminal (“terminal A”) and an exit terminal (“terminal B”). The set of method steps 402 describes that the source samples are received and prepared for the execution of an experiment protocol.

From terminal A (FIG. 4B), the method 400 proceeds to block 408 where the source sample undergoes annotation in which the volume, mass, concentration, and various other pieces of information are recorded pertaining to the source sample. Next, a row (R1) in a table representing the source samples is created. See block 410. At block 412, another row (R2) in the table 346 representing a sample is created storing the key identifier (which is the identifier associated with row R1) and the name of the table representing the source sample. The method 400 proceeds to block 414 where a further row (R3) in the table 348 representing a split batch is created storing the key identifier (which is an identifier representing the row R2 associated with the sample) and the number of split portions. Next, a row (R4) in the table 344 representing a sample unit is created storing the key identifier (which is the unique identifier representing an instantiation of the split batch R3). See block 416. At block 420, the row (R4) in the table 344 representing a sample unit is caused to store the state of the sample. The method then continues to another continuation terminal (“terminal A1”).

From terminal A1 (FIG. 4C), the method 400 proceeds to block 422 where the row (R4) in the table 344 representing a sample unit is caused to store the identifier of the sample derived from the sample unit. Next, the row (R4) in the table 344 representing a sample unit is caused to store an indication of whether the sample unit is a pool (which is represented by the IS_POOL_FLG field 344E). See block 424. At block 426, the row (R4) in the table 344 representing a sample unit is caused to store an indication of whether the sample unit is a member of a pool (which is represented by the IS_POOLUNIT_FLG field 344F). The method then progresses to block 428 where the row (R4) in the table 344 representing a sample unit is caused to store the evaluation status (which is represented by the EVAL_STATUS field 344G). Next, the row (R4) in the table 344 representing a sample unit is caused to store the initial state of the sample (which is represented by the INIT_SAM_TYPSTGID field 344H). See block 430. The method 400 then continues at another continuation terminal (“terminal A2”).

From terminal A2 (FIG. 4D), the method 400 proceeds to block 432 where the row (R4) in the table 344 representing a sample unit is caused to store the current state of the sample (which is represented by the CURR_SAM_TYPSTGID field 344I). Next, the row (R4) in the table 344 representing a sample unit is caused to store the experiment identifier (which is the FUNCTIONAL_UNITID field 344J). See block 434. At decision block 436, a test is made to determine whether there are more source samples for processing. If the answer is YES to the test at decision block 436, the method 400 proceeds to another continuation terminal (“terminal A3”), which loops back to block 408 (FIG. 4B) and repeats the above-discussed processing steps. Otherwise, the answer to the test at decision block 436 is NO, and the method continues to the exit terminal B.

From terminal B (FIG. 4A), the method 400 proceeds to a set of method steps 404, defined between a continuation terminal (“terminal C”) and an exit terminal (“terminal D”). The set of method steps 404 describes the processing of a step in the experiment protocol in which a biological sample and an event connected to its processing history is recorded.

From terminal C (FIG. 4E), the method 400 proceeds to block 438 where a protocol to be executed is selected. Next, all containers of the samples of interest to be used in the execution of the protocol are identified. See block 440. At decision block 442, a test is made to determine whether there are more protocol steps to be performed. If the answer to the test at decision block 442 is NO, the method 400 continues to the exit terminal D. Otherwise, the answer to the test at decision block 442 is YES, and the method 400 continues to block 444 where a protocol step taken from the protocol orders a protocol step 304 to be performed. Next, a portion of the containers of samples of interest associated with the protocol step 304 is identified. See block 446. The validator 306 checks with the database 312 to make sure that the identifiers (such as barcodes) of the portion of the containers are valid. See block 448. The method 400 then continues at another continuation terminal (“terminal C2”).

From terminal C2 (FIG. 4F), the method 400 proceeds to decision block 450 where a test is made to determine whether the identifiers are valid. If the answer to the test at decision block 450 is NO, the method 400 continues to an exit terminal (“terminal F”). If the answer to the test at decision block 450 is YES, the method 400 continues to block 452 where the validator 306 communicates with the workblock to allow it to take action. Next, the workblock communicates with tracking entities 310, such as one or more of the classes illustrated in the class diagram 300. See block 454. The method then continues to another continuation terminal (“terminal C3”).

From terminal C3 (FIG. 4G), the method 400 proceeds to decision block 456 where a test is made to determine whether a new sample is to be created. If the answer to the test at decision block 456 is NO, the method continues at another continuation terminal (“terminal C4”). Otherwise, the answer to the test at decision block 456 is YES, and the method 400 proceeds to block 458 where a tracking entity, such as the class 318, is instantiated and the new sample is created. Next, a row in the table 346 representing a sample is created storing the key identifier of the parent of the new sample. See block 460. (This is where the CAST_TO_SAMPLEID field 344D would be set for a sampleunit that is becoming a sample.) At block 462, also stored in the row in the table 346 representing a sample is the name of the table in which the parent of the new sample is stored. The method 400 then continues to another continuation terminal (“terminal C1”). From terminal C1 (FIG. 4E), the method 400 loops back to decision block 442 where the above-discussed processing steps are repeated.

From terminal C4 (FIG. 4H), the method 400 proceeds to at decision block 464 where a test is made to determine whether a new split batch is to be created. If the answer is NO to the test at decision block 464, the method 400 continues to another continuation terminal (“terminal C5”). Otherwise, the answer to the test at decision block 464 is YES, and the method 400 proceeds to block 466 where a tracking entity representing the new split batch, such as an instantiation of the split batch class 316, is created. Next, a row in the table 348 representing a split batch is created storing the key identifier of the parent of the new split batch. See block 468. At block 470, also stored in the row in the table 348 representing a split batch is a number of split portions. The method 400 then continues to another continuation terminal (“terminal C7”), which loops to block 474 and continues processing. See discussion connected with FIG. 41.

From terminal C5 (FIG. 41), the method proceeds to decision block 472 where a test is made to determine whether a new sample unit is to be created. If the answer to the test at decision block 474 is NO, the method 400 continues at another continuation terminal (terminal C6). Otherwise the answer to the test at decision block 472 is YES and the method 400 proceeds to block 474 where a tracking entity representing the new sample unit, such as an instantiation of the class sample unit 320, is created. Next, a row in the table 344 representing a sample unit is created storing the key identifier of the parent of a new sample unit. See block 476. Also stored in the row in the table representing the sample unit are other pieces of information previously discussed with reference to steps 416-34 (FIGS. 4B-4D). See block 478. The method 400 then continues to terminal C1 where it loops back to decision block 442 and the above-described processing steps are repeated.

From terminal C6 (FIG. 4J), the method 400 proceeds to decision block 480 where a test is made to determine whether a pool is to be created. If the answer to the test at decision block 480 is NO, the method 400 proceeds to the exit terminal D. Otherwise, the answer to the test at decision block 480 is YES, and the method 400 proceeds to block 482 where a tracking entity representing a new pool, such as an instantiation of the pool class 330, is created. (The instantiation of the pool class 330 creates one or more records in the U_POOLUNIT table 350.) Next, a row in the table 344 representing a sample unit is created storing the key identifier of the new pool among other pieces of information (as previously specified from steps 416-34). See block 484. A row in the table 350 representing a pool unit is created for storing the key identifier associated with the new pool as well as key identifiers of the members of the new pool. See block 486. A pool symbolically is an instantiated sampleunit that has poolunit records and the IS_POOL_FLG is set to TRUE. The parents of a pool are sampleunits that become the poolunits that form the pool. The method 400 then continues to the exit terminal D.

From the exit terminal D, the method 400 proceeds to a set of method steps 406, defined between a continuation terminal (“terminal E”) and an exit terminal F. The set of method steps 406 describes that upon request, the state and pedigree of a biological sample is reported to a user.

From terminal E (FIG. 4K), the method 400 proceeds to block 488 where through a user interface, such as a form on a Web page, a user requests to see various pieces of information in connection with a biological sample. (The user interface can be used to enter, modify, or query various pieces of information in connection with a biological sample.) Next, a query is made to the database 312 to obtain a key identifier of the parent. See block 490. At block 492 the result of the query is stored for later reporting. The method 400 then enters decision block 494 where a test is made to determine whether the parent has a parent. If the answer to the test is at decision block 494 is YES, the method 400 loops back to block 490 where the above-described processing steps are repeated. If instead the answer to the test at decision block 494 is NO, the results of the queries are collectively stated in the pedigree of the sample and are reported back to the user. See block 496. The method 400 then proceeds to the exit terminal F and terminates execution.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A system for executing biological experiments, comprising:
a container for containing a biological sample, the container including an identifier that is capable of being scanned and computer-readable to identify the container; and
a sample tracking facility that tracks the biological sample and the container such that various pieces of information in connection with the biological sample are trackable independent of the container that contains the biological sample.

2. The system of claim 1, wherein the biological sample includes deoxyribonucleic acid (DNA), ribonucleic acid (RNA), tissues, lysates, or protein.

3. The system of claim 1, wherein the container includes a plate, a tube, or a slide, each being defined as having one or more wells capable of having a content.

4. The system of claim 1, wherein the system includes a protocol that defines each step in an experiment, the biological sample being processed when the steps of the experiment are executed.

5. The system of claim 4, wherein the sample tracking facility includes a database that stores pieces of information in connection with the biological sample and the processing of the biological sample, the pieces of information including sample pedigree, transformational state, processing stage, quantification data, sample status, sample location, chemical items applied to the biological sample, and equipment applied to the biological sample.

6. A biological sample tracking system, comprising:
a user interface for entering, modifying, or querying the pedigree of a biological sample; and
a database for storing a representation of the biological sample and a history of the processing of the biological sample, the database including a first table set for storing information that pertains to the splitting of the biological sample into multiple portions, the database further including a second table set for storing information that pertains to a pool of the biological sample with another biological sample.

7. The biological sample tracking of claim 6, wherein the first table set stores the identity of the biological sample being split into multiple portions.

8. The biological sample tracking of claim 6, wherein the first table set stores the identities of the multiple portions of the biological sample being split.

9. The biological sample tracking of claim 6, wherein the second table set stores the identity of a third biological sample that is the pool of the biological sample with the another biological sample.

10. The biological sample tracking of claim 6, wherein the second table set stores the identities of the member biological samples comprising the pool.

11. A system for tracking biological samples, comprising:
a number of tracking entities for representing the types of container for containing biological samples and the types of processing of biological samples; and
a number of tables in a database for storing the history of the processing of biological samples, the tables including a first table set for storing identifying information about a biological sample and identifying information about the parent of the biological sample.

12. The system of claim 11, further including a number of work blocks, each work block representing a discrete step of an experiment protocol.

13. The system of claim 12, further including a validator that confirms or denies the validity of various pieces of information associated with a set of containers containing biological samples for a particular work block, the pieces of information including container identities, container types, container processing states, chemical identities, chemical lots, and identities of biological samples.

14. The system of claim 13, further including a work block action component that represents the execution of a work block when the validator has confirmed the validity of various pieces of information associated with the set of containers containing biological samples.

15. The system of claim 14, wherein a set of tracking entities has a corresponding set of tables in the database for accessing the corresponding tables to put data into the tables or to get data out from the tables.

16. A computer-readable medium having one or more data structures stored thereon for use by a computing system to track the history of biological samples, the one or more data structures comprising:
a sample unit class for defining attributes and services connected with a discrete amount of biological sample for experimentation under controlled conditions; and
a container class for defining attributes and services connected with a receptacle for holding biological samples defined by the sample unit class.

17. The one or more data structures of claim 16, further including a pool unit class for defining attributes and services connected with a member of a pool of multiple biological samples.

18. The one or more data structures of claim 16, further including a pool class for defining attributes and services connected with an aggregation of biological samples, the pool class inheriting a set of attributes or services from the sample unit class.

19. The one or more data structures of claim 16, further including a hybridization class for defining attributes and services connected with a hybridization structure of biological samples.

20. The one or more data structures of claim 16, further including a well class for defining attributes and services connected with a well for containing contents.

21. The one or more data structures of claim 16, further including a plate class for defining attributes and services connected with a smooth, flat, thin piece of material on which wells are housed to hold biological samples, the plate class inheriting a set of attributes or services from the container class.

22. The one or more data structures of claim 16, further including a tube class for defining attributes and services connected with a cylindrical structure for holding biological samples, the tube class inheriting a set of attribute or services from the container class.

23. The one or more data structures of claim 16, further including a slide class for defining attributes and services connected with a substrate on which a biological sample is mounted for microscopic examination.

24. The one or more data structures of claim 16, further including a sample class for defining attributes and services connected with a biological sample.

25. The one or more data structures of claim 16, further including a split batch class for defining attributes and services connected with a biological sample that has been divided into multiple portions.

26. The one or more data structures of claim 16, further including a wellcontent class for defining attributes and services connected with an entity representing content in a well.

27. The one or more data structures of claim 16; further including a reagent class for defining attributes and services connected with a substance used as an additive to cause chemical or biological activities in a biological sample.

28. A computer-readable medium having one or more database tables stored thereon for use by a computing system to track, trace, or search pieces of information connected with biological samples, the one or more database tables comprising:
a sample database table that includes:
a sample identifier field that is indicative of a unique identifier of a biological sample that has been instantiated as a record of information in the sample database table;
a parent table name field that is indicative of a name of a database table from which a parent of the biological sample is instantiated as a record of information; and
a parent key identifier field that is indicative of a unique identifier for identifying the parent of the biological sample, which is an instantiation as a record of information.

29. The one or more database tables of claim 28, further comprising a split batch database table that includes:
a split batch identifier field that is indicative of a unique identifier of a split batch that has been instantiated as a record of information in the split batch database;
a sample identifier field that is indicative of an identifier of a biological sample as the parent of the split batch, the biological sample being instantiated as a record of information in the sample database table; and
a number of sample unit field that is indicative of the number of portions that have been split from the biological sample identified by the sample identifier field.

30. The one or more database tables of claim 28, further comprising a sample unit database table that includes:
a sample unit identifier field that is indicative of a unique identifier of a sample unit being instantiated as a record of information in the sample unit database table;
a split batch identifier field that is indicative of a unique identifier of a split batch being instantiated as a record of information in the split batch database table, the split batch being the parent of the sample unit;
a cast to sample identifier field that is indicative of a child sample instantiated as a record of information in the sample database table whose parent is the sample unit;
a pool flag field that is indicative of whether the sample unit is a pool of biological samples;
a pool unit flag field that is indicative of whether the sample unit is a member of a pool of biological samples;
an evaluation status flag field that is indicative of whether the sample unit has passed or failed a quality control test;
an initial sample state identifier field that is indicative of the initial state of the sample unit;
a current sample state identifier field that is indicative of the current state of the sample unit; and
a functional unit identifier field that is indicative of the identifier of an experiment.

31. The one or more database tables of claim 28, further comprising a pool unit relational database table that includes:
a pool unit identifier field that is indicative of a unique identifier of a pool unit or a member of a pool of biological samples;
a pool sample unit identifier field that is indicative of an identifier of the sample unit being instantiated as a record of information in the sample unit database table, the pool sample unit identifier identifying a pool represented by the sample unit;
a member sample unit identifier field that is indicative of an identifier of another sample unit being instantiated as a record of information in the sample unit database table, the member sample unit identifier identifying a member of the pool; and
a member sample state identifier field that is indicative of the state of the sample of a member of the pool being identified by the member sample unit identifier.

32. The one or more database tables of claim 28, further comprising a container database table that includes:
a container identifier field that is indicative of a unique identifier of a container being instantiated as a record of information in the container database table;
a container barcode field that is indicative of a barcode associated with a physical container represented by the container being instantiated as the record of information;
a container type identifier field that is indicative of a container type; and
a protocol container identifier field that is indicative of container type belonging to a protocol.

33. The one or more database tables of claim 28, further comprising a well database table that includes:
a well container identifier field that is indicative of a unique identifier of a well being instantiated as a record of information in the well database table;
a container identifier field that is indicative of a unique identifier of a container being instantiated as a record of information in the container database table;
a well row field that is indicative of a row in the container at which the well resides;
a well column field that is indicative of a column in the container at which the well resides;
a content table name field that is indicative of a name of a database table containing a record of information pertaining to the content of the well;
a content key identifier field that is indicative of an identifier identifying a record of information in the database table that pertains to the content of the well; and
a filled flag field that is indicative of whether the well is filled with biological fragments.

34. A method for tracking biological samples, comprising:
preparing source sample for execution of a protocol;
recording the history of a sample as a step in the protocol is executed; and
reporting the history of the sample to a user upon request by the user.

35. The method of claim 34, wherein recording the history of the sample includes recording a unique identifier of the parent of the sample.

36. The method of claim 35, wherein recording the history of the sample includes recording from which splitbatch the sample portion came.

37. The method of claim 35, wherein recording the history of the sample includes recording whether the sample is a pool or whether the sample is a member of the pool.

38. A computer-readable medium having computer-executable instructions stored thereon that implements a method for tracking biological samples, the method comprising:
preparing source sample for execution of a protocol;
recording the history of a sample as a step in the protocol is executed; and
reporting the history of the sample to a user upon request by the user.

39. The computer-readable medium of claim 38, wherein recording the history of the sample includes recording a unique identifier of the parent of the sample.

40. The computer-readable medium of claim 39, wherein recording the history of the sample includes recording from which splitbatch the sample portion came.

41. The computer-readable medium of claim 39, wherein recording the history of the sample includes recording whether the sample is a pool or whether the sample is a member of the pool.
